(19) United States
(12) Patent Application Publication
(10) Pub. No.: US 2003/0026833 A1
(43) Pub. Date: Feb. 6, 2003

(54) Synergistic antimicrobial textile finish

(76) Inventor: Stephen A. Payne, Charlotte, NC (US)

Correspondence Address: DOUGHERTY & CLEMENTS, TWO FAIRVIEW CENTER, 6230 FAIRVIEW ROAD, SUITE 400, CHARLOTTE, NC, 28210, US

(21) Appl. No.: 10/191,881

(22) Filed: Jul. 9, 2002

Related U.S. Application Data
(60) Provisional application No. 60/304,302, filed on Jul. 10, 2001.

(51) Int. Cl. A61K 9/64

(52) U.S. Cl. 424/460

(57) ABSTRACT

A synergistic antimicrobial textile finish comprising a synergistic antimicrobial combination of antibacterial agent selected from the group consisting of 2,4,4′-trichloro-2′-hydroxy diphenol ether and 2-phenylphenol, or a combination of these, and an antifungal agent of diiodomethyl-4-tolylsulfone, which provides for a synergistic antimicrobial textile finish that is nontoxic, stable, and imparts desired broad spectrum antimicrobial efficacy onto the surface of textile substrates.

### CROSS REFERENCE TO RELATED APPLICATION

This application claims the benefit of U.S. Provisional Application No. 60/304,302, filed Jul. 10, 2001.

### FIELD OF THE INVENTION

This invention relates generally to antimicrobial textile finishes, and more specifically to a textile finish having a synergistic combination of an antibacterial agent and an antifungal agent for biocidal protection of various textile substrates. The present invention is further directed to a textile substrate having a surface that destroys or effectively inhibits the growth of bacteria and fungi thereon by treating the surface of the substrate with an effective amount of the synergistic antimicrobial textile finish.

### BACKGROUND OF THE INVENTION

Textile substrates, when subjected to a moist environment, such as that encountered when textile substrates are in intimate human contact, are susceptible to the growth of microorganisms. The growth of microorganisms on textile substrates may cause fouling, discoloration, deterioration and degradation of the textile substrates. Therefore, various antimicrobial agents have been utilized in conjunction with textile substrates to destroy or effectively inhibit the growth of microorganisms associated therewith.

An antimicrobial agent is a chemical compound that destroys or effectively inhibits the growth of microorganisms. Two major classes of microorganisms are bacteria and fungi. Bacterial microorganisms can be further broken down into gram negative bacteria (i.e., Klebsiella pneumoniae) and gram positive bacteria (i.e., Staphylococcus aureus). Fungal microorganisms (i.e., Aspergillus niger) include yeast, mold, mildew, and algae.

Antimicrobial agents that destroy or effectively inhibit the growth of bacteria are known as antibacterial agents. Chemical compounds that have been associated with various substrates. in the past, to provide antibacterial characteristics, include various antibacterial agents, such as [3-(trimethoxysilyl)propyl]octadecyldimethylammonium chloride; poly(hexamethylenebiguanide) hydrochloride; 10,10′-oxybisphenoxarsine; organic and inorganic silver, copper, and zinc containing compounds; 2,4,4′-trichloro-2′-hydroxy diphenol ether (TRICLOSAN); ester derivatives of 2,4,4′-trichloro-2′-hydroxy diphenol ether; and 2-phenylphenol (DOWICIDE).

Antimicrobial agents that destroy or effectively inhibit the growth of fungi are known as antifungal agents. Chemical compounds that have been incorporated into various substrates in the past, to provide for desired antifungal characteristics, include various types of fungicidal compounds, such as organic antimycotics (i.e., bis(tri-n-butyl tin)oxide, and undecylenic acid); inorganic antimycotics (i.e., mercury(I) chloride, mercury(II) chloride, and silver or copper substituted zeolite); silicon antimycotics (i.e., [3-(trimethoxysilyl)propyl]octadecyldimethyl ammonium chloride); polymeric antimycotics (i.e., polymers containing quaternary ammonium compounds, polymers containing alkylpyridinium compounds, and poly(hexamethylenebiguanide)hydrochloride); azole antimycotics (i.e., bifonazole, clotrimazole, and miconazole); phenolic antimycotics (i.e., 2,2′-methylenebis(4-chlorophenol), and 2,2′-methylenebis(3,4,6-trichlorophenol)); and tolnaftate antimycotics (i.e., 2-naphthyl N-methyl-N-(3-tolyl)thionocarbamate and diiodomethyl-4-tolylsulfone (AMICAL)).

Unfortunately however, currently utilized fungicidal compounds suffer from several drawbacks. First, a number of fungicidal compounds are known to be toxic to humans. Second, many fungicides that are known to inhibit the growth of fungus are only stable when applied to textile substrates under alkaline pH conditions. However, textile finishes are customarily applied to textile substrates at an acidic pH, thus making the fungicide unstable during the application process, which subsequently results in decreased antifungal efficacy of the treated textile substrate. Third, many fungicides that are known to inhibit the growth of fungus are water soluble. Water solubility of the fungicide is inversely proportional to the antifungal durability of the textile substrate having had an antifungal textile finish incorporated thereon. That is, a decreased amount of water soluble antifungal agent remains on the surface of the textile substrate, where antifungal characteristics are exhibited, following each subsequent laundering. Therefore, following each subsequent laundering of the textile substrate, antimicrobial efficacy exhibited on the surface of the textile substrate diminishes.

Therefore a need exists for an antimicrobial textile finish that exhibits nontoxic antibacterial and antifungal protection of various textile substrates, such as fabrics made from synthetic fibers or natural fibers, or blends thereof. Also needed is an antimicrobial textile finish that exhibits stability when applied to various textile substrates under acidic pH conditions and thus retains efficacious antibacterial and antifungal properties during the entire finishing procedure. Also needed is an antimicrobial textile finish that is difficult to remove during standard laundering techniques from the surface of the textile substrate, and to impart desired broad spectrum antibacterial and antifungal protection that is durable throughout the useable life of various textile substrates.

Many known antimicrobial textile finish compositions illustrate synergistic activity against bacterial and fungal microorganisms. As used herein, the term “synergistic” refers to instances where the effectiveness of a composition comprising two or more antimicrobial agents exceeds the sum of the antimicrobial efficacies of the individual antimicrobial agents taken alone. Thus, using a synergistic antimicrobial composition may allow for the use of a lower overall concentration of antimicrobial agent to provide for enhanced antimicrobial efficacy at comparable antimicrobial concentrations.

U.S. Pat. No. 4,830,657 to Jakubowski discloses a synergistic antimicrobial combination comprising 2-bromo-2-bromomethylglutaronitrile and 1,2-benzisothiazoline-3-one. U.S. Pat. No. 4,847,088 to Blank discloses a synergistic antimicrobial composition comprising a mixture of an organosilane quaternary ammonium compound, such as 3-(trimethoxysilyl) propyldimethyloctadecylammonium chloride, and an acid, such as citric, boric, and malic. U.S. Pat. No. 5,700,834 to Stinavage discloses a synergistic antimicrobial composition comprising 1,2-dibromo-2,4-dicyanobutane and an alky guanidine compound. U.S. Pat. No. 5,716,628 to Vinopal discloses a synergistic antimicrobial composition comprising pyrithione acid or a pyrithione salt, or a combination thereof, and at least one compound selected from the group consisting of benzyl and lower alkyl esters of 4-hydroxybenzoic acid, salts thereof, carboxylic acids, salts thereof and combinations thereof. U.S. Pat. No. 5,854,266 to Nelson Jr. discloses a synergistic antimicrobial composition comprising pyrithione acid or a pyrithione salt, or a combination thereof, and an aromatic alcohol selected from the group consisting of phenylethyl alcohol, benzyl alcohol, 2-phenoxyethanol, and combinations thereof. U.S. Pat. No. 5,859,040 to King discloses a synergistic antimicrobial composition comprising 1-hydroxymethylpyrazole and any preservative that will produce a synergistic effect when combined with 1-hydroxymethylpyrazole. U.S. Pat. No. 5,985,934 to Gaffney discloses a synergistic antimicrobial combination comprising 2,4,4′-trichloro-2′-hydroxy diphenol ether and 1,2-dibromo-2,4-dicyanobutane. U.S. Pat. No. 6,008,236 to Oppong discloses a synergistic antimicrobial composition comprising an ionene polymer and a pyrithione salt.

There is a substantial need for an antimicrobial textile finish having enhanced antimicrobial efficacies, which are effective in lower concentrations than historically used. Utilization of lower amounts of antimicrobial agent has a favorable impact on the environment and allows manufactures of antimicrobial textile substrates to realize significant cost savings. In addition, there still remains a need within the textile industry to provide a synergistic antimicrobial textile finish composition that is nontoxic, stable under acidic pH conditions encountered during standard finishing procedures, difficult to remove during repeated standard launderings so as to impart desired broad spectrum antimicrobial efficacy that is durable throughout the useable life of the textile substrate.

### SUMMARY OF THE INVENTION

The present invention is directed to a synergistic antimicrobial textile finish comprising an effective amount of an antibacterial agent of 2,4,4′-trichloro-2′-hydroxy diphenol ether or 2-phenylphenol, or a combination of these, and an effective amount of an antifungal agent of diiodomethyl-4-tolylsulfone.

The present invention is also directed to a dried synergistic antimicrobial textile finish on the surface of a textile substrate comprising an effective amount of the antibacterial agent, and an effective amount of the antifungal agent.

As used herein, the term “effective amount” refers to that amount of a synergistic antimicrobial composition comprising 2,4,4′-trichloro-2′-hydroxy diphenol ether or 2-phenylphenol, or a combination of these, and diiodomethyl-4-tolylsulfone, that is necessary to achieve a desired broad spectrum antimicrobial efficacy, which destroys or effectively inhibits the growth of bacteria and fungi on the surface of the textile substrate.

An essential feature of the present invention is that the effectiveness of the antimicrobial textile finish composition, comprising two or more antimicrobial agents, exceeds the sum of the antimicrobial efficacies of the individual antimicrobial agents when taken alone. That is, by combining the antibacterial agent together with the antifungal agent within the antimicrobial textile finish, a decreased amount of each was necessary to provide for enhanced antibacterial and antifungal efficacy. Therefore, the synergistic antimicrobial textile composition of the present invention allows for the use of a lower overall concentration of antimicrobial agent to provide for enhanced antimicrobial efficacy when compared to the concentrations necessary when either of the individual antibacterial or antifungal agents are employed separately within the textile finish.

The synergistic antimicrobial textile finish is nontoxic and is stable under acidic pH conditions, which are typically encountered during standard finishing procedures for textile substrates. As a result, the synergistic antimicrobial textile finish retains efficacious broad spectrum antifungal and antibacterial properties throughout and subsequent to the entire finishing procedure. Furthermore, the synergistic antimicrobial textile finish is not easily washed away from the surface of the textile fiber substrate during standard laundering techniques, thus imparting durable antibacterial and antifungal protection to various textile substrates.

In the broadest sense, the present invention is an antimicrobial coating on a textile substrate, the coating comprising the dried residue of an antibacterial agent of 2,4,4-trichloro-2-hydroxy diphenol ether or 2-phenylphenol, or a combination of these and an antifungal agent of diiodomethyl-4-tolylsulfone.

Antimicrobial textile finish compositions which are useful for controlling the growth of bacteria and fungi on textile substrates, comprise antibacterial agents and antifungal agents. The antibacterial agents and antifungal agents comprising the antimicrobial textile finish are compatible with various textile substrates, which may be made from a natural fiber or a synthetic fiber, or blends thereof. The antibacterial agents and antifungal agents are also compatible with additives such as, but not limited to, water, dyes, hydrotropes, surfactants, lubricants, preservatives, detergents, and stabilizers. The antibacterial agents and antifungal agents are also stable within aqueous textile systems, which may contain, for example, aqueous organic solutions, emulsions, suspensions, dispersions, concentrates, slurries, coatings, and the like.

### DETAILED DESCRIPTION OF THE PREFERRED EMBODIMENTS

The present invention is directed to an aqueous textile finish having a synergistic antimicrobial composition comprising an effective amount of an antibacterial agent of 2,4,4′-trichloro-2′-hydroxy diphenol ether or 2-phenylphenol, or a combination of these, and an effective amount of an antifungal agent of diiodomethyl-4-tolylsulfone.

The antibacterial agent is present in an amount from about 0.1 weight percent to about 10 weight percent of the finish. The antibacterial agent, 2,4,4′-trichloro-2′-hydroxy diphenol ether (TRICLOSAN), is available from Ciba Specialty Chemicals Corp., Greensboro, N.C., as IRGASAN DP300. The antibacterial agent 2-phenylphenol, otherwise known as, o-phenylphenol (ortho-phenylphenol), is available from Dow Chemical Co., Midland, Mich., as DOWICIDE 1.

The antifungal agent of the finish is present in an amount from about 0.1 weight percent to about 5 weight percent of the finish. The antifungal agent, diiodomethyl-4-tolylsulfone, is available from Angus Chemical Co., Buffalo Grove, Ill., as AMICAL 48.

In addition to water and the synergistic antimicrobial combination (combination) of antibacterial agent and antifungal agent, the finish may also contain one or more additives selected from the group consisting of a dye, a, hydrotrope, a surfactant, a lubricant, a preservative, a detergent, and a stabilizer, or a combination of these. The finish may also be used in diverse liquid formulations, such as aqueous organic solutions, emulsions, suspensions, dispersions, concentrates, slurries, coatings and the like, for treating various textile substrates.

Hydrotropes suitable for use in a finish are selected from the group consisting of xylenesulfonic acid sodium salt, sodium cumene sulfonate, and ammonium cumene sulfonate, or a combination of these. The hydrotrope, when used, is present in an amount from about 1 weight percent to about 15 weight percent of the finish. A preferred hydrotrope of the finish is xylenesulfonic acid sodium salt and is available from Union Carbide Corp., Danbury, Conn., as PILOT SXS-40.

Surfactants suitable for use in a finish are selected from the group consisting of alkyloxypolyethyleneoxyethanol and tridecyl alcohol, or a combination of these. The surfactant is present in an amount from about 3 weight percent to about 20 weight percent of the finish. Tridecyl alcohol contains from about 6 moles to about 9 moles of ethylene oxide. A preferred surfactant of the finish is alkyloxypolyethyleneoxyethanol having the following formula CH₃(CH₂)ₓO(CH₂CH₂O)_{y}CH₂CH₂OH, wherein x has a value of 10 through 14, and it is available from Union Carbide Corp., Danbury, Conn., as TERGITOL 15-S-9.

A suitable lubricant for use in a finish is polyalkylene glycol. The lubricant of the finish is present in an amount from about 3 weight percent to about 20 weight percent of the finish. A preferred lubricant of the finish is polyalkylene glycol having a molecular of weight of about 970 g/mole. The lubricant, polyalkylene glycol, is available from Union Carbide Corp., Danbury, Conn., as UCON 50-HB-260.

A suitable preservative for use in a finish is hexahydro-1,3,5-tris(2-hydroxyethyl)-s-triazine. The preservative of the finish is present in an amount from about 0.1 weight percent to about 5 weight percent of the finish. The preservative is available from Surety Laboratories, North Wales, Pa., as SURCIDE-P.

A suitable detergent for use in a finish is octylphenoxypolyethoxyethanol. The detergent of the finish is present in an amount from about 1 weight percent to about 12 weight percent of the finish. The detergent is available from Union Carbide Corp., Danbury, Conn., as TRITON X-100.

A suitable stabilizer for use in a finish is alkyloxypolyethyleneoxyethanol. The stabilizer of the finish is present in an amount from about 0.5 weight percent to about 9 weight percent of the finish. A preferred stabilizer of the finish is alkyloxypolyethyleneoxyethanol having the following formula CH₃(CH₂)ₓO(CH₂CH₂O)_{y}CH₂CH₂OH, wherein x has a value of 11 through 13. The stabilizer, alkyloxypolyethyleneoxyethanol, is available from Union Carbide Corp., Danbury, Conn., as TERGITOL 15-S-7.

It is to be understood that the antimicrobial agents disclosed herein may be used in combination with other antimicrobial agents. For example, the combination may also contain other bactericides and fungicides in appropriate concentrations and in appropriate instances so as to obtain particularly useful antimicrobial results. Likewise, it is to be understood that the finish disclosed herein may be used in combination with other additives, such as softeners and binders.

The method of producing the finish is accomplished by first solubilizing the antifungal agent, which has an extremely low solubility in water, with a hydrotrope in the presence of water to form a dispersion. Second a surfactant, a lubricant and a preservative may be added to and mixed with the dispersion to form an emulsified antifungal solution. Third, a stabilizer is added to and mixed with the emulsified antifungal solution to form a stable emulsified antifungal solution. Fourth, the antibacterial agent is solubilized in the presence of water, a detergent, and a preservative to form an antibacterial solution. Fifth, the antibacterial solution is added to and mixed with the stable emulsified antifungal solution to form the finish.

The present invention is also directed to a synergistic antimicrobial textile coating on a textile substrate, which produces an antimicrobial textile substrate. The synergistic antimicrobial textile coating comprises a dried residue of an effective amount of an antibacterial agent of 2,4,4′-trichloro-2′-hydroxy diphenol ether or 2-phenylphenol, or a combination of these, and an effective amount of an antifungal agent of diiodomethyl-4-tolylsulfone. The combination of antibacterial agent and antifungal agent are collectively present in an effective amount from about 1 percent fof to about 7 percent fof. The acronym “fof” designates “finish on fabric”, which is a term of art used in the textile industry to represent, in this particular instance, the amount of a dried synergistic antimicrobial textile finish protectively coated on the surface of the substrate. The dried finish imparts nontoxic, stable, and desired broad spectrum antimicrobial protection to the surface of the antimicrobial substrate.

The substrate itself may be made from a synthetic fiber or a natural fiber, or blends thereof. Synthetic fiber is selected from the group consisting of nylon, polyester, polyamide, polyolefin, acrylic and acetate, or blends thereof. Natural fiber is selected from the group consisting of cotton, wool, or hemp, or blends thereof. The natural fiber may be nonwoven, woven, or knit fibers or fabrics. Examples of an antimicrobial article of manufacture having a dried finish protectively coated on a surface of a substrate made from a natural fiber may include, but are not limited to, towels, gym bags, laundry bags, bed linens, carpet, sleeping bags, tents, upholstery, table cloths, lawn furniture, flags, cloth sails, and clothing, such as undergarments, socks, hats, gloves, shoes. It is to be understood that the aforementioned list of antimicrobial articles is not exhaustive however, and that many other articles manufactured from natural fiber may be treated with the finish of the present invention.

### EXAMPLE 1

Coating application of the finish onto a surface of a cotton substrate is accomplished by first maintaining within a reservoir of a jet dyeing machine, an acidic pH from about 5.0 to about 6.0, preferably about 5.5, and at a temperature from about 40° C. to about 70° C., preferably from about 55° C. to about 65° C. Second, the substrate is immersed in the system at a liquor ratio from about 4:1 to about 23:1, preferably from about 7:1 to about 12:1. The liquor ratio is the ratio of the water weight in the system to the dry weight of the substrate prior to being immersed within the system. Third, the finish is added to the system having the substrate immersed therein. Fourth, the finish is coated onto the substrate by maintaining the substrate immersed in the system for about 5 minutes to about 25 minutes, preferably from about 8 minutes to about 16 minutes, until the finish is present on the fiber or substrate at about 1 percent fof to about 7 percent fof, preferably from about 1 percent fof to about 5 percent fof. Fifth, the substrate is removed from the system and then dried to form the antimicrobial substrate. Alternatively, the substrate may be immersed in the system already having the finish present therein.

It is to be understood that the aforementioned preferences are dependent upon a number of factors and system parameters, such as the particular substrate being treated and the particular antibacterial agent and antifungal agent being utilized, as well as their respective concentrations. It is also to be understood that the coating application of the finish onto the surface of the cotton substrate is accomplished by any suitable conventional technique, such as dipping, spraying, and meniscus coating.

### EXAMPLE 2

In order to illustrate the superior antimicrobial activity of the finish, various laboratory experiments were conducted to determine the synergistic antimicrobial efficacy of the substrate that resulted from treating the substrate with the antimicrobial combination of antibacterial agent and antifungal agent. A number of samples were prepared similar to the procedure of Example 1, in which substrates were treated with varied concentrations of antimicrobial agent(s) present within an antibacterial textile finish, an antifungal textile finish, and a synergistic antimicrobial textile finish. The prepared samples were then individually tested for antimicrobial efficacy using American Association of Textile Chemists and Colorists Procedures (AATCC) test method 147 and 30 (III), in which, after a predetermined period of time, the zone of inhibition of the organism (Klebsiella pneumoniae, Staphylococcus aureus, or Aspergillus niger) was measured in millimeters (mm). Results from the laboratory experiments are presented in the form of tabular data in order to facilitate an understanding of the invention.

TABLE 1

| Sample | | | |
|---|---|---|---|
| Identification | K. pneumoniae | S. aureus | A. niger |
| A | 2 mm | 2 mm | 2 mm |
| B | no zone inhibition | no zone inhibition | 2 mm |
| C | 2 mm | 4 mm | 4 mm |

Table 1 shows the results of AATCC test method 147 and 30 (III) for three substrates, namely substrate A, substrate B and substrate C. Each of these substrates were individually placed in nutrient rich broth containing a test organism, selected from the group consisting of Klebsiella pneumoniae, Staphylococcus aureus and Aspergillus niger. Substrates containing Klebsiella pneumoniae and Staphylococcus aureus were incubated at 37° C.±2° C. for 18 to 24 hours. Substrates containing Aspergillus niger were incubated at 28° C.±1° C. for 14 days.

Substrate A was treated with an antibacterial textile finish only. The antibacterial agent was 2,4,4′-trichloro-2′-hydroxy diphenol ether, which is present in an amount of about 0.33 weight percent of the antibacterial textile finish. The hydrotrope of the antibacterial textile finish is xylenesulfonic acid sodium salt; the surfactant of the antibacterial textile finish is alkyloxypolyethyleneoxyethanol having the following formula CH₃(CH₂)ₓO(CH₂CH₂O)_{y}CH₂CH₂OH, wherein x has a value of 10 through 14; the lubricant of the antibacterial textile finish is polyalkylene glycol; the preservative of the antibacterial textile finish is hexahydro-1,3,5-tris(2-hydroxyethyl)-s-triazine; the detergent of the antibacterial textile finish is octylphenoxypolyethoxyethanol; and the stabilizer of the antibacterial textile finish is alkyloxypolyethyleneoxyethanol having the following formula CH₃(CH₂)ₓO(CH₂CH₂O)_{y}CH₂CH₂OH, wherein x has a value of 11 through 13.

Substrate B was treated with an antifungal textile finish only. The antifungal agent of the antifungal textile finish is diiodomethyl-4-tolylsulfone , which is present in an amount from about 0.33 weight percent of the finish. The hydrotrope of the antifungal textile finish is xylenesulfonic acid sodium salt. The surfactant of the antifungal textile finish is alkyloxypolyethyleneoxyethanol having the following formula CH₃(CH₂)ₓO(CH₂CH₂O)_{y}CH₂CH₂OH, wherein x has a value of 10 through 14. The lubricant of the antifungal textile finish is polyalkylene glycol. The preservative of the antifungal textile finish is hexahydro-1,3,5-tris(2-hydroxyethyl)-s-triazine. The detergent of the antifungal textile finish is octylphenoxypolyethoxyethanol, and the stabilizer of the antifungal textile finish is alkyloxypolyethyleneoxyethanol having the following formula CH₃(CH₂)ₓO(CH₂CH₂O)_{y}CH₂CH₂OH, wherein x has a value of 11 through 13.

Substrate C was treated with an antimicrobial textile finish. The antibacterial agent of the antimicrobial textile finish is 2,4,4′-trichloro-2′-hydroxy diphenol ether, which is present in an amount of about 0.33 weight percent of the antimicrobial textile finish. The antifungal agent of the antimicrobial textile finish is diiodomethyl-4-tolylsulfone., which is present in an amount of about 0.33 weight percent of the antimicrobial textile finish. The hydrotrope of the antimicrobial textile finish is xylenesulfonic acid sodium salt. The surfactant of the antimicrobial textile finish is alkyloxypolyethyleneoxyethanol having the following formula CH₃(CH₂)ₓO(CH₂CH₂O)_{y}CH₂CH₂OH, wherein x has a value of 10 through 14. The lubricant of the antimicrobial textile finish is polyalkylene glycol. The preservative of the antimicrobial textile finish is hexahydro-1,3,5-tris(2-hydroxyethyl)-s-triazine. The detergent of the antimicrobial textile finish is octylphenoxypolyethoxyethanol, and the stabilizer of the antimicrobial textile finish is alkyloxypolyethyleneoxyethanol having the following formula CH₃(CH₂)ₓO(CH₂CH₂O)_{y}CH₂CH₂OH, wherein x has a value of 11 through 13.

The aforementioned laboratory experiments illustrate the synergistic antimicrobial efficacy imparted onto the surface of the cotton substrate, which results from treating the surface of the substrate with the finish having a synergistic antimicrobial combination of antibacterial agent and antifungal agent. The surface of the substrate destroys and effectively inhibits the growth of bacteria and fungi thereon. More specifically, the surface of the substrate, which was treated with an effective amount of the finish, was found to be particularly effective, for example, in inhibiting the growth of Klebsiella pneumoniae gram negative bacteria, Staphylococcus aureus gram positive bacteria, and Aspergillus niger fungi. Therefore, it is readily apparent that by combining the antibacterial agent together with the antifungal agent within a textile finish, decreased amounts of each are necessary to provide for enhanced antimicrobial efficacy, as compared to when either of the individual antibacterial or antifungal agents are employed separately within a finish.

The hydrotrope of the antibacterial textile finish is xylenesulfonic acid sodium salt, which is present in an amount from about 1 weight percent to about 15 weight percent of the finish. The surfactant of the antibacterial textile finish is alkyloxypolyethyleneoxyethanol having the following formula CH₃(CH₂)ₓO(CH₂CH₂O)_{y}CH₂CH₂OH, wherein x has a value of 10 through 14, which is present in an amount from about 3 weight percent to about 20 weight percent of the finish. The lubricant of the antibacterial textile finish is polyalkylene glycol, which is present in an amount from about 3 weight percent to about 20 weight percent of the finish. The preservative of the antibacterial textile finish is hexahydro-1,3,5-tris(2-hydroxyethyl)-s-triazine, which is present in an amount from about 0.1 weight percent to about 5 weight percent of the finish. The detergent of the antibacterial textile finish is octylphenoxypolyethoxyethanol, which is present in an amount from about 1 weight percent to about 12 weight percent of the finish. The stabilizer of the antibacterial textile finish is alkyloxypolyethyleneoxyethanol having the following formula CH₃(CH₂)ₓO(CH₂CH₂O)_{y}CH₂CH₂OH, wherein x has a value of 11 through 13, which is present in an amount from about 0.5 weight percent to about 9 weight percent of the finish.

The antifungal agent of the antifungal textile finish is diiodomethyl-4-tolylsulfone; the hydrotrope of the antifungal textile finish is xylenesulfonic acid sodium salt, which is present in an amount from about 1 weight percent to about 15 weight percent of the finish. The surfactant of the antifungal textile finish is alkyloxypolyethyleneoxyethanol having the following formula CH₃(CH₂)ₓO(CH₂CH₂O)_{y}CH₂CH₂OH, wherein x has a value of 10 through 14, which is present is present in an amount from about 3 weight percent to about 20 weight percent of the finish. The lubricant of the antifungal textile finish is polyalkylene glycol, which is present in an amount from about 3 weight percent to about 20 weight percent of the finish. The preservative of the antifungal textile finish is hexahydro-1,3,5-tris(2-hydroxyethyl)-s-triazine, which is present in an amount from about 0.1 weight percent to about 5 weight percent of the finish. The detergent of the antifungal textile finish is octylphenoxypolyethoxyethanol, which is present in an amount from about 1 weight percent to about 12 weight percent of the finish. The stabilizer of the antifungal textile finish is alkyloxypolyethyleneoxyethanol having the following formula CH₃(CH₂)ₓO(CH₂CH₂O)_{y}CH₂CH₂OH, wherein x has a value of 11 through 13, which is present in an amount from about 0.5 weight percent to about 9 weight percent of the finish.

The antibacterial agent of the antimicrobial textile finish is 2,4,4′-trichloro-2′-hydroxy diphenol ether. The antifungal agent of the antimicrobial textile finish is diiodomethyl-4-tolylsulfone, which is present in an amount of about 0.33 weight percent of the antimicrobial textile finish. The hydrotrope of the antimicrobial textile finish is xylenesulfonic acid sodium salt, which is present in an amount from about 1 weight percent to about 15 weight percent of the finish. The surfactant of the antimicrobial textile finish is alkyloxypolyethyleneoxyethanol having the following formula CH₃(CH₂)ₓO(CH₂CH₂O)_{y}CH₂CH₂OH, wherein x has a value of 10 through 14, which is present is present in an amount from about 3 weight percent to about 20 weight percent of the finish. The lubricant of the antimicrobial textile finish is polyalkylene glycol, which is present in an amount from about 3 weight percent to about 20 weight percent of the finish. The preservative of the antimicrobial textile finish is hexahydro-1,3,5-tris(2-hydroxyethyl)-s-triazine, which is present in an amount from about 0.1 weight percent to about 5 weight percent of the finish. The detergent of the antimicrobial textile finish is octylphenoxypolyethoxyethanol, which is present in an amount from about 1 weight percent to about 12 weight percent of the finish. The stabilizer of the antimicrobial textile finish is alkyloxypolyethyleneoxyethanol having the following formula CH₃(CH₂)ₓO(CH₂CH₂O)_{y}CH₂CH₂OH, wherein x has a value of 11 through 13, which is present in an amount from about 0.5 weight percent to about 9 weight percent of the finish.

It is to be clearly understood that the foregoing description and specific embodiments are merely illustrative of the best mode of the present invention and the principles thereof, and are not intended as limitations on the scope of the invention, and therefore various modifications and additions may be made to the structures, compounds, compositions, and methods described herein by those skilled in the art, without departing substantially from the essential concepts, spirit and scope of the invention.

What is claimed is:

1. An antimicrobial aqueous-based textile finish comprising:
i) water;
ii) an antibacterial agent selected from the group consisting of 2,4,4′-trichloro-2′-hydroxy diphenol ether or 2-phenylphenol, or a combination of these, and
iii) an effective amount of an antifungal agent comprising diiodomethyl-4-tolylsulfone.

2. The finish of claim 1, wherein said antibacterial agent comprises 0.1-10% by weight of said finish.

3. The finish of claim 1, wherein said antifungal agent comprises 0.1-5% by weight of said finish.

4. The finish of claim 3, wherein said antibacterial agent comprises 0.1-10% by weight of said finish.

5. The finish of claim 1, wherein said finish further comprises one or more additives selected from the group consisting of a dye, a hydrotrope, a surfactant, a lubricant, a preservative, a detergent, a stabilizer, or a combination of thereof.

6. The finish of claim 5, wherein said hydrotrope is xylenesulfonic acid sodium salt; said surfactant is alkyloxypolyethyleneoxyethanol having the formula CH₃(CH₂)ₓO(CH₂CH₂O)_{y}CH₂CH₂OH, wherein x has a value of 10 through 14; said lubricant is polyalkylene glycol; said preservative is hexahydro-1,3,5-tris(2-hydroxyethyl)-s-triazine; said detergent is octylphenoxypolyethoxyethanol; and said stabilizer is alkyloxypolyethyleneoxyethanol having the following formula CH₃(CH₂)ₓO(CH₂ CH₂O)_{y}CH₂CH₂OH, wherein x has a value of 11 through 13.

7. The finish of claim 6, wherein said hydrotrope is present in a range of about 1-15 weight percent of said finish; said surfactant is present in a range of about 3-20 weight percent of said finish; said lubricant is present in a range of about 3-20 weight percent of said finish; said preservative is present in a range of about 0.1-5 weight percent of said finish; said detergent is present in a range of about 1-12 weight percent of said finish; and said stabilizer is present in a range of about 0.5-9 weight percent of said finish.

8. The finish of claim 5, wherein said hydrotrope is present in a range of about 1-15 weight percent of said finish; said surfactant is present in a range of about 3-20 weight percent of said finish; said lubricant is present in a range of about 3-20 weight percent of said finish; said preservative is present in a range of about 0.1-5 weight percent of said finish; said detergent is present in a range of about 1-12 weight percent of said finish; and said stabilizer is present in a range of about 0.5-9 weight percent of said finish.

9. An antimicrobial coating on a textile substrate, said antimicrobial coating comprising: a dried residue of: i) an antibacterial agent of 2,4,4′-trichloro-2′-hydroxy diphenol ether or 2-phenylphenol, or a combination of these, and ii) an antifungal agent of diiodomethyl-4-tolylsulfone.

10. The antimicrobial coated substrate according to claim 9, wherein said residue is present in an amount from about 1 weight percent to about 5 weight percent on said textile substrate.

11. An antimicrobial coated substrate according to claim 9, wherein said textile substrate is selected from the group consisting of synthetic fibers or natural fibers, or blends thereof.

12. An antimicrobial coated substrate according to claim 11, wherein said synthetic fiber is selected from the group consisting of nylon, polyester, polyamide, polyolefin, acrylic and acetate, or blends thereof.

13. An antimicrobial coated substrate according to claim 11, wherein said natural fiber is selected from the group consisting of cotton, wool, or hemp, or blends thereof.

14. An antimicrobial coated substrate according to claim 13, wherein said natural fiber may be nonwoven, woven, or knit fibers or fabrics.

15. The antimicrobial coated substrate of claim 9, wherein said antibacterial agent comprises 0.1-10% by weight of said finish.

16. The antimicrobial coated substrate of claim 9, wherein said antifungal agent comprises 0.1-5% by weight of said finish.

17. The antimicrobial coated substrate of claim 16, wherein said antibacterial agent comprises 0.1-10% by weight of said finish.

18. The antimicrobial coated substrate of claim 9, wherein said finish also comprises one or more additives selected from the group consisting of a dye, a hydrotrope, a surfactant, a lubricant, a preservative, a detergent, and a stabilizer, or a combination of these.

19. The antimicrobial coated substrate of claim 18, wherein said hydrotrope is xylenesulfonic acid sodium salt; said surfactant is alkyloxypolyethyleneoxyethanol having the following formula CH₃(CH₂)ₓO(CH₂CH₂O)_{y}CH₂CH₂OH, wherein x has a value of 10 through 14; said lubricant is polyalkylene glycol; said preservative is hexahydro-1,3,5-tris(2-hydroxyethyl)-s-triazine; said detergent is octylphenoxypolyethoxyethanol; and said stabilizer is alkyloxypolyethyleneoxyethanol having the following formula CH₃(CH₂)ₓO(CH₂CH₂O)_{y}CH₂CH₂OH, wherein x has a value of 11 through 13.

20. The antimicrobial coated substrate of claim 19, wherein said hydrotrope is present in a range of about 1-15 weight percent of said finish; said surfactant is present in a range of about 3-20 weight percent of said finish; said lubricant is present in a range of about 3-20 weight percent of said finish; said preservative is present in a range of about 0.1-5 weight percent of said finish; said detergent is present in a range of about 1-12 weight percent of said finish; and said stabilizer is present in a range of about 0.5-9 weight percent of said finish.

21. The antimicrobial coated substrate of claim 18, wherein said hydrotrope is present in a range of about 1-15 weight percent of said finish; said surfactant is present in a range of about 3-20 weight percent of said finish; said lubricant is present in a range of about 3-20 weight percent of said finish; said preservative is present in a range of about 0.1-5 weight percent of said finish; said detergent is present in a range of about 1-12 weight percent of said finish; and said stabilizer is present in a range of about 0.5-9 weight percent of said finish.
